(19) United States
(12) Patent Application Publication
(10) Pub. No.: US 2026/0115463 A1
(43) Pub. Date: Apr. 30, 2026

(54) CLOSED-LOOP BRAIN-COMPUTER INTERFACE TIBIAL NEUROMODULATION SYSTEM

(71) Applicants: CHINA REHABILITATION SCIENCE INSTITUTE, Beijing (CN); CHINA REHABILITATION RESEARCH CENTER, Beijing (CN)

(72) Inventor: Limin LIAO, Beijing (CN)

(73) Assignees: CHINA REHABILITATION SCIENCE INSTITUTE, Beijing (CN); CHINA REHABILITATION RESEARCH CENTER, Beijing (CN)

(21) Appl. No.: 19/336,615

(22) Filed: Sep. 23, 2025

(30) Foreign Application Priority Data
Oct. 28, 2024 (CN) 202411506480.9

(51) Int. Cl. A61N 1/36; A61B 5/00; A61B 5/374

(52) U.S. Cl. CPC A61N 1/36007; A61B 5/374; A61B 5/725; A61N 1/36031; A61N 1/36153

(57) ABSTRACT

The present disclosure relates to a closed-loop brain-computer interface tibial neuromodulation system, including a scalp electrophysiological monitoring terminal, a tibial neuromodulation terminal, and a programming terminal. The scalp electrophysiological monitoring terminal is configured to monitor a scalp electrophysiological signal in real-time via a non-invasive manner, and to identify an abnormal signal indicative of bladder overactivity upon an occurrence of a urinary urgency symptom of a patient. The tibial neuromodulation terminal is configured to, in response to detecting the abnormal signal, initiate a tibial nerve electrical stimulation to achieve an on-demand neuromodulation for the patient during a symptom onset and persistence period of the bladder overactivity. The programming terminal is configured to obtain a treatment parameter related to the on-demand neuromodulation to initiate a real-time tibial nerve electrical stimulation for the patient.

### CROSS-REFERENCE TO RELATED APPLICATIONS

This application claims priority to Chinese patent application No. 202411506480.9, filed on Oct. 28, 2024, the entire contents of which are incorporated herein by reference.

### TECHNICAL FIELD

The present disclosure generally relates to the field of nerve electrical stimulation, and in particular, to a closed-loop brain-computer interface tibial neuromodulation system.

### BACKGROUND

A tibial neuromodulation stimulation (TNS) or tibial neuromodulation (TNM) is an effective manner to treat overactive bladder (OAB) (urinary frequency, urinary urgency, and urgent urinary incontinence) by stimulating the tibial nerve at the Sanyinjiao acupoint in conjunction with Chinese medicine acupoint theory. The tibial neuromodulation is generally performed by employing a tibial nerve electrical stimulation generator that applies electrical stimulation pulses to the posterior tibial nerve at Sanyinjiao acupoint by transcutaneous patch or percutaneous needle puncture. A principle of the tibial neuromodulation is that the tibial nerve contains nerve fibers from L4 to S3, which originate from the same spinal cord segment as the nerve fibers that control the bladder and pelvic floor. By stimulating somatic afferent components to inhibit bladder afferent activity, blocking transmission of abnormal sensation to the spinal cord and brain, and then affecting and regulating behavior of sacral nerve-controlled effector organs such as the bladder, urethral sphincter, and pelvic floor, and plays a role of “neuromodulation” to treat overactive bladder.

At present, a general treatment plan for tibial neuromodulation treatment for overactive bladder is to select a frequency in a range of 0-200 Hz and a constant intensity electrical stimulation pulse for a treatment process of 30 minutes to 1 hour based on an actual clinical treatment situation. Although this general treatment plan has achieved certain clinical results, it still has problems such as poor real-time performance, poor treatment accuracy, and inability to achieve “on-demand” stimulation or treatment.

Therefore, it is desired to propose a closed-loop brain-computer interface tibial neuromodulation system that can monitor a symptom of bladder overactivity in real time and accurately, and promptly initiate a tibial nerve electrical stimulation during a symptom onset and persistence period, thereby achieving an on-demand stimulation and a precision treatment, and intervening the transmission of abnormal urination sensation to the spinal cord and brain, and effectively relieving the symptom of the patient.

### SUMMARY

The present disclosure provides a closed-loop brain-computer interface tibial neuromodulation system. The closed-loop brain-computer interface tibial neuromodulation system includes: a scalp electrophysiological monitoring terminal, a tibial neuromodulation terminal, and a programming terminal. The scalp electrophysiological monitoring terminal is connected to the tibial neuromodulation terminal, and the programming terminal is connected to the tibial neuromodulation terminal. The scalp electrophysiological monitoring terminal is configured to monitor a scalp electrophysiological signal in real-time via a non-invasive manner, and to identify an abnormal signal indicative of bladder overactivity upon an occurrence of a urinary urgency symptom of a patient. The scalp electrophysiological signal indirectly characterizes frequency bands related to the bladder overactivity upon the occurrence of the urinary urgency symptom of the patient. The tibial neuromodulation terminal is configured to, in response to detecting the abnormal signal, initiate a tibial nerve electrical stimulation to achieve an on-demand neuromodulation for the patient during a symptom onset and persistence period of the bladder overactivity. The programming terminal is configured to obtain a treatment parameter related to the on-demand neuromodulation. The closed-loop brain-computer interface tibial neuromodulation system further comprises: configuration information. The configuration information is configured to be generated by the programming terminal and transmitted to the scalp electrophysiological monitoring terminal. The scalp electrophysiological monitoring terminal is further configured to identify an existence of the abnormal signal via the scalp electrophysiological signal monitored in real-time and the configuration information. The configuration information at least includes feature set configuration information and a feature identification model. The feature set configuration information is configured to screen a processing result of the scalp electrophysiological signal. The feature identification model is configured to identify whether the abnormal signal exists based on the processing result of the scalp electrophysiological signal. The tibial neuromodulation terminal is further configured to, in response to determining that the abnormal signal reverts to a non-abnormal signal indicative of normal bladder activity, automatically terminate the tibial nerve electrical stimulation according to a preset delay time. The programming terminal is further configured to obtain input information entered by a doctor and generate the configuration information based on the input information. The programming terminal includes a feature identification setting unit, the scalp electrophysiological monitoring terminal includes a feature identification unit. The feature identification setting unit is configured to receive model input information within the input information, generate the feature identification model based on the model input information, and transmit the feature identification model to the feature identification unit. The programming terminal includes a feature value set setting unit, the scalp electrophysiological monitoring terminal includes a feature value set configuration unit. The feature value set setting unit is configured to receive set input information within the input information, generate the feature set configuration information based on the set input information, and transmit the feature set configuration information to the feature value set configuration unit. The feature value set configuration unit is configured to screen the processing result of the scalp electrophysiological signal according to the feature set configuration information and determine a feature value set. The feature value set at least includes feature values under an overactive bladder during an awake activity state of the patient and feature values under the overactive bladder during a transitional sleep state of the patient. The feature identification unit is configured to identify the feature value set based on the feature identification model and determine whether the abnormal signal exists.

### BRIEF DESCRIPTION OF THE DRAWINGS

The present disclosure may be further illustrated by way of exemplary embodiments, which will be described in detail through the accompanying drawings. Those embodiments are not limiting, and in those embodiments, the same numbering denotes the same structure. In the accompanying drawings:

FIG. 1 is a schematic diagram illustrating a structure of a closed-loop brain-computer interface tibial neuromodulation system according to some embodiments of the present disclosure;

FIG. 2 is a schematic diagram illustrating energy states of electroencephalogram features of a healthy person in different bladder states according to some embodiments of the present disclosure; and

FIG. 3 is a schematic diagram illustrating waveforms of a processed and identified electroencephalogram features of a healthy person in different bladder states according to some embodiments of the present disclosure.

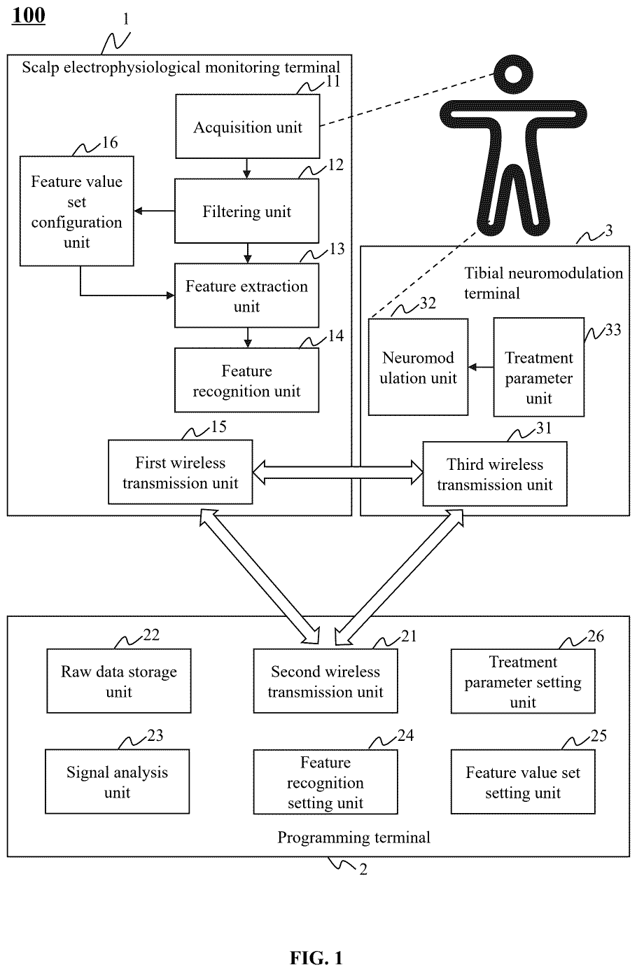

In the FIGS. 1-3: 1, scalp electrophysiological monitoring terminal; 2, programming terminal; 3, tibial neuromodulation terminal; 11, acquisition unit; 12, filtering unit; 13, feature extraction unit; 14, feature identification unit; 15, first wireless transmission unit; 16, feature value set configuration unit; 21, second wireless transmission unit; 22, raw data storage unit; 23, signal analysis unit; 24, feature identification setting unit; 25, feature value set setting unit; 26, treatment parameter setting unit; 31, third wireless transmission unit; 32, neuromodulation unit; 33, treatment parameter unit.

### DETAILED DESCRIPTION

To more clearly illustrate the technical solutions of the embodiments of the present disclosure, the accompanying drawings required to be used in the description of the embodiments are briefly described below. Obviously, the accompanying drawings in the following description are only some examples or embodiments of the present disclosure, and it is possible for those skilled in the art to apply the present disclosure to other similar scenarios under these drawings without creative labor. The present disclosure may be applied to other similar scenarios based on these drawings without creative labor. Unless obviously obtained from the context or the context illustrates otherwise, the same numeral in the drawings refers to the same structure or operation.

It should be understood that the terms “system,” “device,” “unit,” and/or “module” as used herein are used to distinguish between different components, elements, parts, sections, or assemblies at different levels. However, these terms may be replaced by other expressions if other words accomplish the same purpose.

As shown in the present disclosure and in the claims, unless the context clearly suggests an exception, the words “one,” “a,” “an,” and/or “the” do not refer specifically to the singular, but may also include the plural.

To make the purpose, technical solutions, and advantages of the present disclosure clearer, the technical solutions of the present disclosure will be clearly and completely described below in conjunction with the specific embodiments of the present disclosure and the corresponding accompanying drawings. Obviously, the described embodiments are only a portion of the embodiments of the present disclosure, and not all the embodiments. Based on the embodiments of the present disclosure, all other embodiments obtained by those skilled in the art without making creative labor fall within the scope of protection of the present disclosure.

To facilitate understanding of a technical solution of a closed-loop brain-computer interface tibial neuromodulation system of the embodiments of the present disclosure, a technical principle of the embodiments of the present disclosure is first explained.

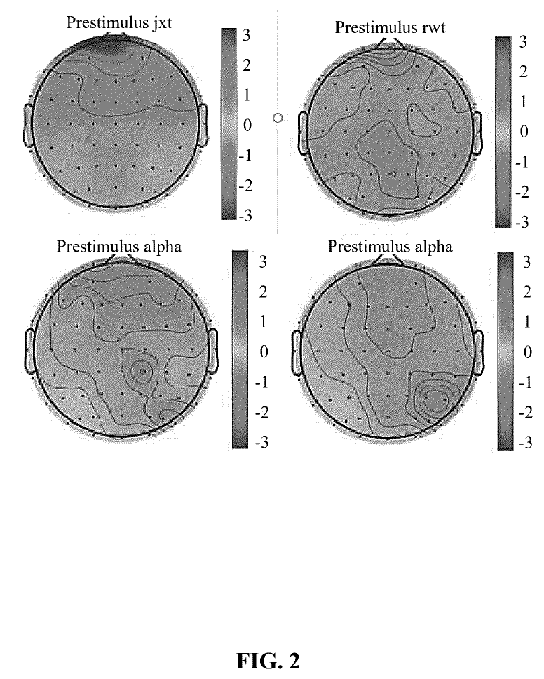

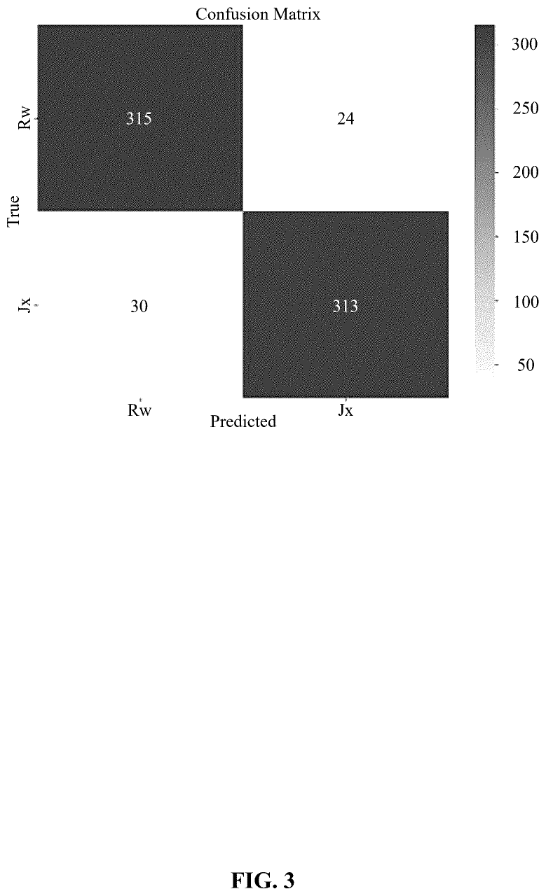

Inventors measured scalp electrophysiological signals of different persons in a bladder-empty state and a urinary urgency state, respectively. Referring to FIG. 2 and FIG. 3, employing a healthy person as an example, study results after a plurality of experiments showed that under the urinary urgency state, prefrontal α waves of a subject are significantly lower than the prefrontal α waves of the subject under the bladder-empty state. Combined with FIG. 2, it can be seen that the left side indicates energies of α waves reflecting an electroencephalogram (EEG) state of the subject under the bladder-empty state, and the right side indicates energies of α waves reflecting the electroencephalogram state of the subject under the urinary urgency state. Afterwards, the inventors process and analyze collected electroencephalogram data by a machine learning technique. Combined with FIG. 3, it can be seen that for 343 segments of a resting state (i.e., the bladder-empty state), 313 segments are identified as a resting state and 30 segments are identified as a task state; and for 339 segments of a task state (i.e., the urinary urgency state), 24 segments are identified as a resting state and 315 segments are identified as a task state. It can be seen that there exists a significant difference in the electroencephalogram features of the subject during different bladder states.

Based on this, the technical concept of the present disclosure is to monitor, based on an electroencephalogram monitoring technology, a scalp electrophysiological signal under a urinary urgency symptom of a patient and a scalp electrophysiological signal under a non-urinary urgency symptom of the patient via a non-invasive manner; identify an abnormal signal related to bladder overactivity based on a relationship between the scalp electrophysiological signals and the brain function condition of the patient; then initiate a tibial nerve electrical stimulation at a symptom onset of the urinary urgency symptom based on an electroencephalogram activity change of the patient, to achieve an on-demand neuromodulation and a precise treatment during a symptom onset and persistence period, thereby achieving a technical effect of improving the real-time and intelligence level of the system, improving treatment accuracy, and effectively alleviating the symptom of the overactive bladder of the patient.

The following is a detailed description of the technical solutions provided by various embodiments of the present disclosure in conjunction with the accompanying drawings.

FIG. 1 is a schematic diagram illustrating a structure of a closed-loop brain-computer interface tibial neuromodulation system according to some embodiments of the present disclosure.

In some embodiments, as shown in FIG. 1, the closed-loop brain-computer interface tibial neuromodulation system includes: a scalp electrophysiological monitoring terminal 1, a programming terminal 2, and a tibial neuromodulation terminal 3. The scalp electrophysiological monitoring terminal 1 is connected to the tibial neuromodulation terminal 3, and the programming terminal 2 is connected to the tibial neuromodulation terminal 3. The scalp electrophysiological monitoring terminal 1 is configured to monitor a scalp electrophysiological signal in real-time via a non-invasive manner, and to identify an abnormal signal indicative of bladder overactivity upon an occurrence of a urinary urgency symptom of a patient. The scalp electrophysiological signal indirectly characterizes frequency bands related to the bladder overactivity upon the occurrence of the urinary urgency symptom of the patient. The tibial neuromodulation terminal 3 is configured to, in response to detecting the abnormal signal, initiate a tibial nerve electrical stimulation to achieve an on-demand neuromodulation for the patient during a symptom onset and persistence period of the bladder overactivity. The programming terminal 2 is configured to obtain a treatment parameter related to the on-demand neuromodulation.

The non-invasive manner refers to a manner that does not involve surgery or other means of invading the human body. For example, the non-invasive manner may include body surface electrode signal acquisition, in vitro processing and analysis, a conventional wet electrode acquisition manner, a semi-dry electrode acquisition manner, a dry electrode acquisition manner, a non-contact acquisition manner, or the like.

The scalp electrophysiological signal refers to a signal on a surface of the scalp that reflects electrical activity in the brain. For example, the scalp electrophysiological signal may include θ waves, α waves, and β waves. The θ wave refers to an electrophysiological signal of the brain in a meditative state. The α wave refers to an electrophysiological signal of the brain in an awake but relaxed state. The β wave refers to an electrophysiological signal of the brain in an awake but tense state.

In some embodiments, the scalp electrophysiological signal includes a change in an electrophysiological signal caused by an electroencephalogram signal induced on the scalp by related physiological and psychological activity caused by the overactive bladder. The change of the scalp electrophysiological signal may include a change of the electrophysiological signal caused by emotional anxiety, urination sensation, and related movement.

The patient refers to a person who employs the closed-loop brain-computer interface tibial neuromodulation system to regulate the bladder of the person.

The abnormal signal refers to a signal related to the bladder overactivity. The bladder overactivity may include urinary frequency, urinary urgency, and urgent urinary incontinence.

In some embodiments, the closed-loop brain-computer interface tibial neuromodulation system is adapted for monitoring, symptom recognition, assessment, and treatment of an OAB patient (a patient with the overactive bladder). Of course, the technical solution of the present disclosure may be extended to the monitoring and treatment of other symptoms of urinary tract dysfunction, and is not limited herein.

In some embodiments, the abnormal signal includes an abnormal signal related to the bladder overactivity, such as a somatosensory evoked potential, an emotion evoked potential, and an action evoked potential that induces a urination response. The somatosensory evoked potential refers to a potential signal related to a somatosensory state of the patient, such as urinary urge. The emotion evoked potential refers to a potential signal related to an emotional state, such as anxiety and worry, which is triggered by the external environment when the patient has the urinary urge. The action evoked potential refers to a potential signal related to an action state, which is triggered when the patient has the urinary urge.

In some embodiments, the above somatosensory evoked potential, emotion evoked potential, and action evoked potential are all related to the functional activity of the brain of the patient. Under different bladder states, the electroencephalogram features of different potentials have significant differences. In some embodiments, a processor may determine whether there exists an abnormality in the scalp electrophysiological signal of the patient by monitoring and identifying the somatosensory evoked potential, the emotion evoked potential, the action evoked potential, or the like. For example, the processor may generate a time-domain diagram based on the processing of the scalp electrophysiological signal of the patient, and determine specific frequency bands of the bladder overactivity related to the urinary urgency symptom of the patient to screen abnormal spectral energy and a change state.

Of course, the above contents regarding the classification, identification, judgment, or the like of the abnormal signals are exemplary embodiments, which are only for the purpose of facilitating understanding and simplifying the description, and are not to be construed as limitations on the present disclosure.

In some embodiments, the processor may determine whether there exists an abnormality in the scalp electrophysiological signal of the patient by monitoring and identifying the somatosensory evoked potential, the emotion evoked potential, the action evoked potential, or the like.

For example, the processor may generate the time-domain diagram based on the processing of the scalp electrophysiological signal of the patient, and determine the specific frequency bands of the bladder overactivity related to the urinary urgency symptom of the patient to screen abnormal spectral energy and a change state, and determine a scalp electrophysiological signal corresponding to the at least one of the abnormal spectral energy or the change state as the abnormal signal.

The time-domain diagram refers to a diagram of the scalp electrophysiological signal changing over time. The specific frequency bands refer to scalp electrophysiological signals under the different brain states. The spectral energy refers to an energy distribution of the scalp electrophysiological signals. The change state refers to a change in the frequency, amplitude, and energy of the scalp electrophysiological signal.

In some embodiments, the processor may determine whether signals in different frequency bands of the scalp electrophysiological signal are abnormal, and if a signal in at least one specific frequency band of the scalp electrophysiological signal shows an abnormality, the scalp electrophysiological signal is considered as the abnormal signal. For example, for the θ waves in the scalp electrophysiological signal, if a short-time spectral energy of the θ waves exceeds an energy threshold (e.g., 0.6), the signal in the frequency band is identified as abnormal. For the α waves in the scalp electrophysiological signal, if a short-time spectral energy of the α waves is below an energy threshold (e.g., 0.4), the signal in the frequency band is identified as abnormal. For the β waves in the scalp electrophysiological signal, if a short-time spectral energy change rate of the β waves exceeds an energy threshold (e.g., 0.2), the signal in the frequency band is identified as abnormal. If the signal in any frequency band of the θ waves, the α waves, or the β waves is identified as abnormal, the scalp electrophysiological signal at that moment is determined to be the abnormal signal. The energy threshold refers to a maximum or minimum value of specific spectral energy under a normal condition. In some embodiments, the energy threshold may be preset by the processor based on a default setting.

In some embodiments, the closed-loop brain-computer interface tibial neuromodulation system further includes configuration information. The configuration information is configured to be generated by the programming terminal 2 and transmitted to the scalp electrophysiological monitoring terminal 1. The scalp electrophysiological monitoring terminal 1 is further configured to identify an existence of the abnormal signal via the scalp electrophysiological signal monitored in real-time and the configuration information. For more description of the programming terminal 2 may be found in the related descriptions below.

A feature identification model refers to a model that is used to identify the existence of an abnormal signal based on a processing result of the scalp electrophysiological signal. In some embodiments, the feature identification model may be a machine learning model. For example, the feature identification model includes a machine learning model such as a support vector machine, a neural network, or the like.

Feature value set configuration information refers to a parameter that is used to screen a specific feature value.

The configuration information refers to information that is used to determine whether the abnormality exists in the scalp electrophysiological signal.

In some embodiments, the configuration information at least includes feature set configuration information and the feature identification model.

The feature set configuration information is configured to screen the processing result of the scalp electrophysiological signal.

The feature identification model is configured to identify whether an abnormal signal exists based on the processing result of the scalp electrophysiological signal.

In some embodiments, the configuration information may be generated by the programming terminal 2 and transmitted to the scalp electrophysiological monitoring terminal. More descriptions regarding the generation of the configuration information by the programming terminal 2 may be found in the related descriptions below.

In some embodiments, the processor may screen the processing result of the scalp electrophysiological signal based on the feature set configuration information, and extract feature values to determine a feature value set for the screened processing result of the scalp electrophysiological signal; determine, based on the feature value set, whether the abnormal signal exists in the scalp electrophysiological signal via the feature identification model.

In some embodiments, an input of the feature identification model may include the feature value set, and an output of the feature identification model may include whether the abnormal signal exists.

In some embodiments, the feature identification model may be obtained by training based on a plurality of first training samples with first training labels.

In some embodiments, a first training sample may include a sample feature value set. The first training sample may be obtained based on historical data. For example, the historical data may include a historical feature value set.

In some embodiments, the first training label may be the existence or absence of the abnormal signal in the scalp electrophysiological signal corresponding to the first training sample. The first training label may be labeled by the processor and/or manually based on the historical data. For example, the processor and/or a technician may analyze a large number of pieces of historical data to determine whether the abnormal signal exists in the scalp electrophysiological signal corresponding to the historical feature value set and label the first training label.

In some embodiments, the processor may input the first training sample into an initial feature identification model, construct a loss function based on whether an abnormal signal exists output from the initial feature identification model and the first training label, and update the initial feature identification model based on the loss function. When a preset condition is met, the training of the initial feature identification model is completed, and a trained feature identification model is obtained. The preset condition may be that the loss function converges, a count of iterations reaches a threshold, or the like.

The feature value set refers to a set of feature values of the processing result of the scalp electrophysiological signal. For example, the feature value set may include spectral energy, a variation, etc.

For example, the processor may extract the feature values of the scalp electrophysiological signal and screen the feature values for the scalp electrophysiological signal based on the feature set configuration information, and determine the scalp electrophysiological signal corresponding to the feature values that satisfy the feature set configuration information as a screened scalp electrophysiological signal.

In some embodiments, the scalp electrophysiological monitoring terminal 1 may include an acquisition unit 11, a filtering unit 12, and a feature extraction unit 13. The acquisition unit 11 may be configured to acquire the scalp electrophysiological signal located on a head via a scalp-mounted electrode. The feature extraction unit 13 may be configured to perform cluster analysis on the scalp electrophysiological signal processed by the filtering unit 12 according to the specific frequency band, and calculate the feature values within the specific frequency band as the processing result of the scalp electrophysiological signal.

In some embodiments, the acquisition unit 11 is configured to acquire the scalp electrophysiological signal and transmit the scalp electrophysiological signal to the filtering unit 12. Acquisition positions include the parietal lobe located directly above the head and frontal lobe located at the forehead, etc., and are not limited here.

In some embodiments, the specific frequency band includes a short-time spectral energy and a change rate of the θ waves within a 3.5 Hz to 8 Hz bandpass, a short-time spectral energy and a change rate of the α waves within an 8 Hz to 12 Hz bandpass, and a short-time spectral energy and a change rate of the β waves within a 12 Hz to 33 Hz bandpass.

In some embodiments, the filtering unit 12 performs a filtering process and a Fourier transform on the scalp electrophysiological signal and transmits the scalp electrophysiological signal after processing to the feature extraction unit 13. The filtering process may remove an interfering signal unrelated to a target signal, such as a high-frequency signal and an eye electrophysiological signal in the scalp electrophysiological signal, and filter out a low-frequency signal and a high-frequency signal by a band-pass filter, retaining a signal in the frequency band of 3 Hz to 35 Hz.

In some embodiments, the feature extraction unit 13 performs the cluster analysis on the scalp electrophysiological signal processed by the filtering unit 12 according to the specific frequency band, calculates the feature value within the specific frequency band, and designates the feature value within the specific frequency band as the processing result of the scalp electrophysiological signal.

In some embodiments, the specific frequency band for performing the cluster analysis includes the short-time spectral energy and the change rate of the θ waves within the 3.5 Hz to 8 Hz bandpass, the short-time spectral energy and the change rate of the α waves within the 8 Hz to 12 Hz bandpass, and the short-time spectral energy and the change rate of the β waves within the 12 Hz to 33 Hz bandpass.

In some embodiments, the feature extraction unit 13 may perform the cluster analysis on the scalp electrophysiological signal processed by the filtering unit 12 according to the specific frequency band.

For example, the feature extraction unit 13 may classify the scalp electrophysiological signal processed by the filtering unit 12 according to the specific frequency band, discretize scalp electrophysiological signal of each specific frequency band into a plurality of discrete points; integrate all of the discrete points into a same plane, and perform spatial density cluster on the plurality of discrete points in the plane to form a plurality of clusters, each cluster representing a set of discrete points in a similar position, and determine a geometric center of each cluster; and determine a plurality of feature values based on the geometric centers of the plurality of clusters.

The feature values within the specific frequency band refer to features of the scalp electrophysiological signal within the specific frequency band. In some embodiments, the feature values may include the geometric center of the cluster, the density of the cluster, or the like.

In some embodiments, the feature extraction unit 13 may determine the feature values within the specific frequency band according to time-domain analysis.

In some embodiments, the feature extraction unit 13 may designate the feature values within the specific frequency band computed after the time-domain analysis as the processing result of the scalp electrophysiological signal.

In some embodiments, by designing the acquisition unit and the feature extraction unit, the scalp electrophysiological monitoring terminal can more accurately acquire and process the scalp electrophysiological signal, improve the accuracy of the identification of the abnormal signal, realize individualized monitoring, more accurately extract signal features related to each specific frequency band, improve the accuracy of the identification of the abnormal signal, and more comprehensively reflect activity features of the brain under different states, thereby more accurately identifying the abnormal signals.

In some embodiments, the programming terminal 2 includes a feature value set setting unit 25, and the scalp electrophysiological monitoring terminal 1 may further include a feature identification unit 14 and a feature value set configuration unit 16.

In some embodiments, the processor may summarize the processing result of the scalp electrophysiological signal determined by the feature extraction unit 13 and store it in the feature value set configuration unit 16.

In some embodiments, the feature value set configuration unit 16 is configured to screen the feature value set from the processing result of the scalp electrophysiological signal.

In some embodiments, the feature identification unit 14 is configured to identify the feature value set and determine whether the abnormal signal exists. The feature identification unit 14 may include a feature identification model.

In some embodiments, the identification of the feature value set includes, but is not limited to, adopting a variety of identification methods such as Bayesian algorithms, cluster analysis, neural network classification, and so on in machine learning, and the identification effect can be evaluated by metrics such as matching degree, identification rate, and false identification rate, etc., which will not be repeated here.

In some embodiments, the feature value set setting unit 25 may be configured to receive set input information within input information, generate the feature set configuration information based on the set input information, and transmit the feature set configuration information to the feature value set configuration unit 16. The feature value set configuration unit 16 is configured to screen the processing result of the scalp electrophysiological signal and determine the feature value set based on the feature set configuration information.

For example, the feature value set configuration unit 16 may screen the processing result of the scalp electrophysiological signal based on the feature set configuration information to obtain the feature value set and input the feature value set into the feature identification model of the feature identification unit 14. The feature identification model determines whether the feature value set is abnormal, and then determines whether the scalp electrophysiological signal corresponding to the feature value set is an abnormal signal.

In some embodiments, the scalp electrophysiological monitoring terminal 1 may further be configured to determine, based on pre-wearing data, a stable period and a fluctuating period; in response to determining that the patient is in the fluctuating period, employ a first preset algorithm to determine, based on the scalp electrophysiological signal, the processing result of the scalp electrophysiological signal and physiological monitoring data, whether the abnormal signal exists; and in response to determining that the patient is in the stable period, employ a second preset algorithm to determine whether the abnormal signal exists.

The pre-wearing data refers to a scalp electrophysiological signal acquired over a predefined period. In some embodiments, the pre-wearing data may also include a signal-to-noise ratio of the scalp electrophysiological signals acquired over the predefined period.

In some embodiments, the scalp electrophysiological monitoring terminal 1 may acquire the pre-wearing data via the electrode.

The stable period refers to a period corresponding to a scalp electrophysiological signal with a large signal-to-noise ratio.

The fluctuating period refers to a period corresponding to a scalp electrophysiological signal with a small signal-to-noise ratio.

In some embodiments, the processor may determine the stable period and the fluctuating period based on the pre-wearing data and a stable threshold.

For example, the processor may identify a period in the pre-wearing data corresponding to a scalp electrophysiological signal with a signal-to-noise ratio that is less than the stable threshold as the fluctuating period, and a period in the pre-wearing data corresponding to a scalp electrophysiological signal with a signal-to-noise ratio that is greater than or equal to the stable threshold as the stable period. The stable threshold refers to a minimum value of a signal-to-noise ratio of a relatively stable scalp electrophysiological signal. In some embodiments, the stable threshold may be preset by the processor based on default settings.

As another example, the processor may also construct a signal-to-noise ratio (SNR) time-segmented feature library based on the pre-wearing data, and perform a comparison among the SNR time-segmented feature library based on the scalp electrophysiological signal that is currently obtained to determine whether a current time point is in the fluctuating period or the stable period. The SNR time-segmented feature library may include a correspondence between the scalp electrophysiological signal, the signal-to-noise ratio, and whether the scalp electrophysiological signal is in the fluctuating period. In some embodiments, the SNR time-segmented feature library may be constructed by the processor based on default parameters, or by the technician based on the historical data.

The first preset algorithm refers to an algorithm for determining whether an abnormal signal exists in the scalp electrophysiological signal in response to determining that the patient is in the fluctuating period. The first preset algorithm may be a first fluctuating abnormality model. The first fluctuating abnormality model may be a machine learning model. For example, the first fluctuating abnormality model may be a convolutional neural network (CNN), etc.

In some embodiments, an input of the first fluctuating abnormality model may include the scalp electrophysiological signal, the processing result of the scalp electrophysiological signal, and the physiological monitoring data, and an output of the first fluctuating abnormality model may include whether the abnormal signal exists.

The physiological monitoring data refers to data related to physical features of the patient. For example, the physiological monitoring data may include a sweat frequency, a sweat volume, a heart rate variability, a skin electrical response, or the like.

In some embodiments, the processor may obtain the physiological monitoring data in various ways. For example, the processor may obtain the sweat frequency and the sweat volume via a humidity sensor, and obtain the heart rate variability and the skin electrical response via a smart wearable device (e.g., a smart bracelet, a smart watch, etc.), and a skin electrical response sensor.

In some embodiments, the training of the first fluctuating abnormality model is similar to the training of the feature identification model, and the relevant content may be found in the previous descriptions, which will not be repeated here.

The second preset algorithm refers to an algorithm for determining whether an abnormal signal exists in the scalp electrophysiological signal in response to determining that the patient is in the stable period.

For example, the second preset algorithm may be an algorithm for performing a comparative analysis based on the scalp electrophysiological feature set during the pre-wearing usage and a feature value set of an electrophysiological signal monitored during a formal wearing usage.

As another example, the second preset algorithm may be an algorithm for determining, based on the processing result of the scalp electrophysiological signal, whether the abnormal signal exists by querying a first preset table. The first preset table may include a relationship between the processing result of the scalp electrophysiological signal and whether the scalp electrophysiological signal is an abnormal signal. In some embodiments, the first preset table may be preset by the processor based on default parameters.

In some embodiments, the first preset algorithm is more complex than the second preset algorithm. For example, the first preset algorithm requires the scalp electrophysiological signal, the processing result of the scalp electrophysiological signal, and the physiological monitoring data, while the second preset algorithm requires only the processing result of the scalp electrophysiological signal, or only the scalp electrophysiological feature set during the pre-wearing usage and the electrophysiological signal monitored during the formal wearing usage. A count of pieces of data processed by the first preset algorithm is significantly larger than a count of pieces of data processed by the second preset algorithm. A model of the first preset algorithm needs to be trained iteratively; the first preset table corresponding to the second preset algorithm may be preset, and the feature value set may be obtained in real time, and a computational load of the second preset algorithm is smaller than a computational load of the first preset algorithm.

In some embodiments, the system divides a monitoring period into the stable period and the fluctuating period based on the pre-wearing data, and employs different algorithms to identify the abnormal signal for different periods, which can more accurately identify the abnormal signal, improve therapeutic effects, reduce misjudgments, and enhance the comfort of the patient. This design not only enhances the adaptability and flexibility of the system but also improves treatment accuracy and patient compliance.

In some embodiments, the scalp electrophysiological monitoring terminal 1 is further configured to extract first scalp electrophysiological feature values under a non-overactive bladder during an awake activity state and a transitional sleep state of the patient through the pre-wearing usage during the predefined period; extract second scalp electrophysiological feature values under the overactive bladder during the awake activity state and the transitional sleep state of the patient; determine the scalp electrophysiological feature set for the pre-wearing usage based on the first scalp electrophysiological feature values and the second scalp electrophysiological feature values; and perform the comparative analysis between the scalp electrophysiological feature set during the pre-wearing usage and the feature value set of the electrophysiological signal monitored during the formal wearing usage, to determine whether a requirement for initiating an electrical stimulation therapy is met to perform an individualized pre-configuration for the patient.

The predefined period refers to a period during which the patient performs pre-wearing. The pre-wearing refers to an operation in which the patient wears the scalp electrophysiological monitoring terminal before a formal treatment.

In some embodiments, the predefined period may be preset by the processor based on the default parameters.

A daily activity state refers to an activity state in which the patient is awake and not stimulated.

A semi-sleep state refers to a state in which the patient closes eyes and is not fully asleep.

The non-overactive bladder refers to a symptom of the patient when the bladder overactivity does not exist.

In some embodiments, the processor may identify physical features under a normal condition of the patient as the non-overactive bladder.

A first scalp electrophysiological feature value refers to a feature value of the first scalp electrophysiological signal. The first scalp electrophysiological signal refers to a scalp electrophysiological signal under the non-overactive bladder. For example, the scalp electrophysiological signal may include a scalp electrophysiological signal during the awake activity state and a scalp electrophysiological signal during the transitional sleep state of the patient under the non-overactive bladder.

The overactive bladder refers to a symptom of the patient when the bladder overactivity exists.

In some embodiments, the processor may identify physical features of the patient under the bladder overactivity as the overactive bladder.

The second scalp electrophysiological feature value refers to a feature value of the second scalp electrophysiological signal. The second scalp electrophysiological signal refers to a scalp electrophysiological signal of the patient under the overactive bladder. For example, the second scalp electrophysiological signal may include a scalp electrophysiological signal during the awake activity state and a scalp electrophysiological signal during the transitional sleep state of the patient under the overactive bladder.

In some embodiments, the processor may obtain the first scalp electrophysiological signal under the non-overactive bladder during the awake activity state and the transitional sleep state of the patient, extract feature values of a first scalp electrophysiological signal, and determine the feature values of the first scalp electrophysiological signal as the first scalp electrophysiological feature values. The processor may obtain the second scalp electrophysiological signal under the overactive bladder during the awake activity state and the transitional sleep state of the patient, extract feature values of a second scalp electrophysiological signal, and determine the feature values of the second scalp electrophysiological signal as the second scalp electrophysiological feature values. A manner for determining the first scalp electrophysiological feature values and the second scalp electrophysiological feature values may be found elsewhere in the present disclosure (e.g., the manner for determining the feature value and related descriptions thereof), and will not be repeated herein.

The scalp electrophysiological feature set refers to a set of the first scalp electrophysiological feature values or the second scalp electrophysiological feature values. In some embodiments, the scalp electrophysiological feature set may include a first scalp electrophysiological feature set and a second scalp electrophysiological feature set.

In some embodiments, the processor may obtain a plurality of first scalp electrophysiological feature values of the patient under the non-overactive bladder and a plurality of second scalp electrophysiological feature values of the patient under the overactive bladder, and integrate the plurality of first scalp electrophysiological feature values into the first scalp electrophysiological feature set, and integrate the plurality of second scalp electrophysiological feature values into the second scalp electrophysiological feature set.

In some embodiments, the processor may extract the feature value set of the electrophysiological signal monitored during the formal wearing usage, and perform the comparative analysis between the scalp electrophysiological feature set during the pre-wearing usage and the feature value set of the electrophysiological signal monitored during the formal wearing usage, to determine whether the requirement for initiating the electrical stimulation therapy is met to perform the individualized pre-configuration for the patient.

For example, the processor may calculate a similarity degree between the first scalp electrophysiological feature set during the pre-wearing usage and the feature value set during the formal wearing usage based on the first scalp electrophysiological feature set during the pre-wearing usage and the feature value set of the electrophysiological signal monitored during the formal wearing usage. The processor may calculate a similarity degree between the second scalp electrophysiological feature set during the pre-wearing usage and the feature value set during the formal wearing usage based on the second scalp electrophysiological feature set during the pre-wearing usage and the feature value set of the electrophysiological signal monitored during the formal wearing usage. The processor may determine a feature value in the feature value set during the formal wearing usage corresponding to the second scalp electrophysiological feature set with a similarity degree greater than a similarity threshold as an abnormal feature value. The processor may determine a scalp electrophysiological signal corresponding to the abnormal feature value as the abnormal signal. The processor may determine that the current state of the patient meets the requirement for initiating the electrical stimulation therapy in response to determining the existence of the abnormal signal during the formal wearing usage. The similarity threshold refers to a minimum similarity degree at which the scalp electrophysiological feature set during the pre-wearing usage is similar to the feature value set of the electrophysiological signal monitored during the formal wearing usage. In some embodiments, the similarity threshold may be preset by the processor based on the default settings.

In some embodiments, the scalp electrophysiological monitoring terminal 1 may directly determine the abnormal signal and perform the electrical stimulation therapy. For example, the scalp electrophysiological monitoring terminal 1 may determine whether there exists the abnormality in the scalp electrophysiological signals of the patient by monitoring and identifying the somatosensory evoked potential, the emotion evoked potential, the action evoked potential, etc., and initiate the electrical stimulation therapy in response to determining that the abnormality exists in the scalp electrophysiological signal of the patient.

More descriptions regarding the scalp electrophysiological monitoring terminal determining whether there exists an abnormality in the scalp electrophysiological signals may be found in the previous related descriptions.

In some embodiments, the scalp electrophysiological monitoring terminal 1 may perform the individualized pre-configuration for the patient to improve the treatment accuracy. The pre-configuration refers to a process that personalizes the closed-loop brain-computer interface tibial neuromodulation system based on the first scalp electrophysiological feature values and the second scalp electrophysiological feature values obtained during the pre-wearing of the patient. In some embodiments, the processor may integrate the first scalp electrophysiological feature values and the second scalp electrophysiological feature values into the first scalp electrophysiological feature set and the second scalp electrophysiological feature set, respectively; determine a pre-configuration result (e.g., the model input information of the feature identification model, etc.) based on the first scalp electrophysiological feature set and the second scalp electrophysiological feature set, and perform the pre-configuration on the system based on the pre-configuration result.

For example, when a monitoring treatment is required, the doctor may configure, based on a clinical condition of the patient or the pre-configuration result (e.g., the model input information) of the scalp electrophysiological monitoring terminal 1, the feature identification model to the scalp electrophysiological monitoring terminal 1 in advance by a feature identification setting unit 24. The processor configures, based on the set input information of the doctor, the first scalp electrophysiological feature set, and the second scalp electrophysiological feature set, the feature set configuration information by the feature value set setting unit 25, and performs the pre-configuration on the system based on the feature identification model that is configured and the feature set configuration information. The clinical condition of the patient refers to the current health state or a symptom of the patient. The pre-configuration result refers to the result of performing the individualized pre-configuration. The pre-configuration result may include the model input information of the feature identification model, the treatment parameter, or the like.

The set input information refers to a parameter input by the doctor for configuring the feature value set. In some embodiments, the input information may include the set input information.

Of course, the above descriptions related to the pre-configuration are only an exemplary description and should not be construed as a limitation of the present disclosure.

In addition, the scalp electrophysiological monitoring terminal 1 of the embodiments of the present disclosure may be used in conjunction with the programming terminal 2, and, of course, it may be improved by a person of relevant skill in the art to realize offline use of the scalp electrophysiological monitoring terminal 1. For example, a standardized treatment parameter corresponding to a relevant symptom is pre-stored in the scalp electrophysiological monitoring terminal 1, and the tibial neuromodulation terminal 3 is regulated based on the information that is pre-stored, which is not limited here. More descriptions regarding the programming terminal 2 may be found in the related descriptions below.

In some embodiments, extracting the scalp electrophysiological feature values during different states of the patient through the pre-wearing usage during the predefined period, and performing the individualized pre-configuration based on the feature values, can generate individualized monitoring and treatment plans for each patient, improving the pertinence and effectiveness of the treatment. The system can dynamically adjust the monitoring and treatment parameters according to the actual state of the patient, improving the adaptability and flexibility of the system.

In some embodiments, the programming terminal 2 is configured to obtain the treatment parameter related to the on-demand neuromodulation.

In some embodiments, the programming terminal 2 may run on an electronic device such as a PC, a tablet, or a cell phone. In some embodiments, the programming terminal 2 may include a processor. The processor may process data from one or more modules of the closed-loop brain-computer interface tibial neuromodulation system, or an external data source. The processor may include a central processing unit (CPU), an application-specific integrated circuit (ASIC), an application-specific instruction set processor (ASIP), an image processing unit (GPU), a physical operations processing unit (PPU), a digital signal processor (DSP), a processor, a microcontroller unit, a reduced instruction set computer (RISC), a microcontroller, or the like, or any combination thereof. In some embodiments, the processor may be local or remote. The processor may be integrated into the one or more modules of the closed-loop brain-computer interface tibial neuromodulation system, or may be a separate module of the closed-loop brain-computer interface tibial neuromodulation system.

The on-demand neuromodulation refers to a process of adjusting a tibial nerve electrical stimulation parameter based on the scalp electrophysiological signal.

The treatment parameter refers to a parameter that adjusts the tibial nerve electrical stimulation.

In some embodiments, the treatment parameter includes at least one of: stimulation waveform, amplitude, pulse width, frequency, rise cycle, fall cycle, duration cycle, treatment duration, frequency modulation or constant frequency, high frequency, low frequency, voltage, or current source.

In some embodiments, the treatment parameter related to the on-demand neuromodulation may be a standardized treatment parameter that is pre-stored in a treatment parameter setting unit 26, may be treatment parameter automatically generated, based on the electroencephalogram signal of the patient, by a machine learning model that is pre-constructed, or may be an electrical stimulation pulse parameter that is comprehensively judged and set by the doctor based on interactive interface information of a display unit, which may not be limited herein.

In some embodiments, the programming terminal 2 may also be configured to determine the treatment parameter in response to determining that the abnormal signal exists in the scalp electrophysiological signal based on monitoring processing information.

The monitoring processing information refers to data of the scalp electrophysiological signal that is pre-processed, and the processing result.

In some embodiments, the monitoring processing information may include the scalp electrophysiological signal, the processing result of the scalp electrophysiological signal, and an abnormal signal determination result.

In some embodiments, the programming terminal 2 may determine the treatment parameter (e.g., the frequency, the amplitude, the pulse width, etc.) based on an abnormal frequency of a historical abnormal signal determination result. The historical abnormal signal determination result may be an abnormal signal determination result during a period before the current moment. The abnormal frequency refers to an occurring frequency of the abnormal signal during the period before the current moment.

For example, the programming terminal 2 may determine the treatment parameter based on the abnormal frequency of the historical abnormal signal determination result by querying a second preset table. The second preset table may include a relationship between the abnormal frequency and the treatment parameter. In some embodiments, the second preset table may be preset by the programming terminal 2 based on the default settings, or by the doctor based on experience.

As another example, the programming terminal 2 may determine, based on the abnormal frequency of the historical abnormal signal determination result and historical recovery time, the treatment parameter by querying a third preset table. The recovery time refers to a time when a bladder overactivity state reverts to a non-bladder overactivity state. The third preset table may include a relationship between the abnormal frequency, the historical recovery time, and the treatment parameter. In some embodiments, the third preset table may be preset by the programming terminal 2 based on the default settings, or by the doctor based on the experience.

In some embodiments, the abnormal frequency and the recovery time are positively correlated with the treatment parameter.

In some embodiments, the programming terminal 2 may also be configured to determine the treatment parameter based on the monitoring processing information and user data.

The user data refers to basic information about the patient. For example, the user data may include age, gender, weight, disease history, etc., of the patient.

In some embodiments, the programming terminal 2 may obtain the user data from a storage device.

In some embodiments, the programming terminal 2 may determine the treatment parameter based on the monitoring processing information, and adjust the treatment parameter based on the user data and a preset rule. The preset rule refers to a predetermined rule for adjusting the treatment parameter. In some embodiments, the preset rule may be preset by the programming terminal 2 based on the default settings, or by the doctor based on the experience.

For example, if the patient is older than 60 years of age, the amplitude in the treatment parameter is decreased by 10%; if the patient is younger than 30 years of age, the frequency in the treatment parameter is increased by 5%; if the patient's weight exceeds 80 kg, the pulse width in the treatment parameter is increased by 15%; if the patient's weight is below 50 kg, the amplitude in the treatment parameter is decreased by 20%; if the patient has a disease history record with ineffective previous treatment, the frequency in the treatment parameter is changed to a frequency modulation mode (e.g., the frequency changes in a range of 20 Hz-40 Hz); if the patient has a disease history record with low stimulus tolerance, the pulse width in the treatment parameter is fixed to a minimum value (e.g., 100 μs).

Further, the scalp electrophysiological monitoring terminal 1 also transmits information such as the abnormal signal determination result to the programming terminal 2. The doctor makes a comprehensive judgment based on the information displayed at the programming terminal 2, gives a corresponding treatment parameter, and inputs the treatment parameter via the treatment parameter setting unit 26. The programming terminal 2 transmits the treatment parameter to the tibial neuromodulation terminal 3. This enables the doctor to configure an individualized treatment parameter based on the above displayed information in a targeted manner, effectively enhancing the treatment effect.

In some embodiments, by determining the treatment parameter based on the monitoring processing information and the user data, the programming terminal 2 can fully consider the actual situation of the user, adjust the electrical stimulation parameter in an individualized manner, and improve the treatment effect.

In some embodiments, by determining the treatment parameter based on the monitoring processing information, the programming terminal 2 can more accurately regulate the electrical stimulation to ensure that the treatment plan is consistent with the current physiological state of the patient.

In some embodiments, in response to determining that the scalp electrophysiological signal is in the fluctuating period, the programming terminal 2 may determine the treatment parameter based on patient state information, the user data, the physiological monitoring data, and a symptom severity. More descriptions regarding the fluctuating period may be found in the previous related descriptions.

The patient state information refers to a current activity state of the patient. For example, the patient state information may include resting, walking, exercising, sleeping, or the like.

In some embodiments, the programming terminal 2 may determine the patient state information via the smart wearable device. The smart wearable device may include the smart bracelet, the smart watch, etc.

The symptom severity may be expressed by a numerical value. The higher the numerical value is, the more serious the symptom of the patient is.

In some embodiments, the programming terminal 2 may construct a symptom feature vector based on abnormal data and pathological data, and determine the symptom severity by matching a vector database.

The abnormal data refers to data related to the abnormal signal. For example, the abnormal data may include a frequency of occurrence of the abnormal signal, a duration of the abnormal signal, or the like.

In some embodiments, the programming terminal 2 may determine the abnormal data based on historical monitoring processing information in a statistical manner.

The pathological data refers to data related to the symptoms of the patient. For example, the pathological data may include data such as blood glucose, blood fat, inflammatory factors (e.g., CRP, etc.), etc., of the patient.

In some embodiments, the programming terminal 2 may obtain medical examination data of the patient via the storage device, thereby obtaining the pathological data of the patient.

The vector database may be constructed based on historical abnormal data, historical pathological data, and a historical symptom severity by the programming terminal 2.

In some embodiments, the programming terminal 2 may determine, based on the patient state information, the user data, the physiological monitoring data, and the symptom severity, the treatment parameter through a parameter determination model.

The parameter determination model refers to a model used to determine the treatment parameter.

In some embodiments, the parameter determination model may be a machine learning model. For example, the parameter determination model may include a CNN, etc.

In some embodiments, an input of the parameter determination model may include the patient state information, the user data, the physiological monitoring data, and the symptom severity. An output of the parameter determination model may include the treatment parameter.

In some embodiments, the parameter determination model may be obtained by training based on a plurality of second training samples with second training labels.

In some embodiments, a second training sample may include sample patient state information, sample user data, sample physiological monitoring data, and a sample symptom severity. The second training sample may be obtained based on the historical data. For example, the sample patient state information, the sample user data, the sample physiological monitoring data, and the sample symptom severity may be historical patient state information, historical user data, historical physiological monitoring data, and the historical symptom severity in the historical data, respectively.

In some embodiments, the second training label may be a historical treatment parameter corresponding to the second training sample. The second training label may be labeled by the processor and/or manually based on the historical data. For example, the processor and/or the technician may perform statistics on a large amount of historical data to obtain the treatment parameter used on the patient, and label the treatment parameter used on the patient as the second training label.

In some embodiments, the programming terminal 2 may input the second training sample into an initial parameter determination model, construct a loss function based on the treatment parameter output from the initial parameter determination model and the second training label, and update the initial parameter determination model based on the loss function. When a preset condition is met, the training of the initial parameter determination model is completed, and the trained parameter determination model is obtained. The preset condition may be that the loss function converges, a count of iterations reaches a threshold, or the like.

In some embodiments, determining the treatment parameter based on the parameter determination model is beneficial to improving the accuracy of the treatment parameter, dynamically adjusting the treatment parameter based on the physiological state monitored in real-time, generating the individualized treatment parameter for each patient, and ensuring that the treatment plan always meets current physiological needs of the patient, thereby improving the treatment effect.

In some embodiments, the programming terminal 2 is further configured to obtain the input information entered by the doctor and generate the configuration information based on the input information.

The input information refers to an instruction or data entered by the doctor through the programming terminal 2.

In some embodiments, the programming terminal 2 further includes the feature identification setting unit 24, and the scalp electrophysiological monitoring terminal 1 includes the feature identification unit 14. The feature identification setting unit 24 is configured to receive the model input information within the input information, generate the feature identification model based on the model input information, and transmit the feature identification model to the feature identification unit 14. For example, the programming terminal 2 may set, by the feature identification setting unit 24, a parameter, such as a model parameter and a threshold for screening the abnormal signal, and transmit the parameter to the feature identification unit 14 to complete the identification of the abnormal signal.

More descriptions regarding the feature identification unit 14 may be found in the related descriptions below.

In some embodiments, the input information of the doctor includes the model input information.

The model input information refers to a parameter entered by the doctor for generating and configuring the feature identification model.

In some embodiments, the programming terminal 2 may determine the feature identification model based on the model input information. For example, the programming terminal 2 may determine the model input information based on the input information entered by the doctor, and determine a model parameter (e.g., a learning rate, a tier level, a count of nodes, a count of iterations, etc.) based on the model input information, and generate the feature identification model based on the model parameter.

In some embodiments, the programming terminal 2 may transmit the generated feature identification model to the feature identification unit 14. For example, the programming terminal 2 may transmit the feature identification model to the feature identification unit 14 via a second wireless transmission unit 21.

In some embodiments, the feature value set configuration unit 16 may be further configured to screen the processing result of the scalp electrophysiological signal according to the feature set configuration information and determine the feature value set. The feature value set includes at least the feature values under the overactive bladder during the awake activity state of the patient and the feature values under the overactive bladder during the transitional sleep state of the patient.

For example, the feature value set may include information such as a spectral energy and a change rate, a peak value, a root-mean-square (RMS) value, a duration, etc. The feature value set may be a subset of a raw processing result set or the raw processing result set itself. In some embodiments, the feature value set configuration unit 16 may only include a portion of pieces of data within the feature value set setting unit 25.

In some embodiments, the programming terminal 2 further includes a raw data storage unit and a signal analysis unit; the raw data storage unit is configured to receive and display the scalp electrophysiological signal, the processing result of the scalp electrophysiological signal, and the abnormal signal determination result; and the signal analysis unit is configured to process the scalp electrophysiological signal and output and display a programming-processed signal.

For example, reference to FIG. 1, the programming terminal 2 includes the raw data storage unit 22, the signal analysis unit 23, the feature identification setting unit 24, the feature value set setting unit 25, and the treatment parameter setting unit 26. In some embodiments, the processing of the scalp electrophysiological signal by the signal analysis unit 23 includes the filtering process and the Fourier transform.

In some embodiments, the doctor may input corresponding input information into the programming terminal 2 based on information displayed by the raw data storage unit 22 and the signal analysis unit 23, thereby generating the configuration information of the treatment parameter and transmitting the configuration information to the scalp electrophysiological monitoring terminal 1.

It should be noted that since the filtering process and the Fourier transform of the programming terminal 2 are similar to the processing steps of the filtering unit 12 and the feature extraction unit 13 of the scalp electrophysiological monitoring terminal 1, which will not be repeated here. It is understood that both the scalp electrophysiological monitoring terminal 1 and the programming terminal 2 may process raw data that is acquired, and the difference lies in arithmetic power, i.e., the scalp electrophysiological monitoring terminal 1 may process only a portion of data after screening and identify the portion of data to improve a computing speed of the system.

The programming-processed signal refers to a scalp electrophysiological signal processed by the programming terminal 2.

In some embodiments, the programming terminal 2 may display information such as the scalp electrophysiological signal, the processing result of the scalp electrophysiological signal, and a programming-processed signal result in the form of a chart, thereby facilitating the doctor to perform an individualized and precise configuration for the patient. For example, the programming terminal 2 may receive, in real time, scalp electrophysiological signal data transmitted by the scalp electrophysiological monitoring terminal 1, and display a time-domain feature, a frequency-domain feature, and a time-frequency feature of the data in the form of a chart. Meanwhile, the programming terminal 2 also calculates the feature values of the scalp electrophysiological signal in a way similar to that of the scalp electrophysiological monitoring terminal 1, and displays the feature values of the scalp electrophysiological signal in a chart along with a signal processing result from the scalp electrophysiological monitoring terminal 1. With the help of the chart and the data, the doctor can configure an individual-specific identification standard for the patient by the feature identification setting unit 24 and the feature value set setting unit 25 to improve the treatment accuracy.

In some embodiments, by including the raw data storage unit and the signal analysis unit, the programming terminal may receive and display the scalp electrophysiological signal and the processing result of the scalp electrophysiological signal, provide a data visualization function, and enhance the intelligence and adaptability of the system. The scalp electrophysiological signal and the processing result of the scalp electrophysiological signal stored in the raw data storage unit may be used for subsequent research and analysis to help optimize the treatment plan, and by analyzing the stored data, the system can continuously improve a signal processing algorithm and a determination manner of the treatment parameter to improve overall performance of the system.

In some embodiments, the programming terminal 2 further includes a treatment parameter setting unit 26. The treatment parameter setting unit 26 is configured to, in response to determining that the raw data storage unit 22 receives the scalp electrophysiological signal, the processing result of the scalp electrophysiological signal, and the abnormal signal determination result, receive the treatment parameter that is individualized input by the doctor; and transmit the treatment parameter to the tibial neuromodulation terminal 3 to achieve the on-demand neuromodulation for the patient during the symptom onset and persistence period of the bladder overactivity according to the treatment parameter after automatic initiation of the tibial nerve electrical stimulation.

For example, in response to determining that the scalp electrophysiological monitoring terminal 1 monitors the existence of the abnormal signal in the scalp electrophysiological signal, determine the patient is under the overactive bladder, and the programming terminal 2 may initiate the electrical stimulation when the abnormal signal appears for the first time, and continue to initiate the electrical stimulation when the patient is under the overactive bladder.

More descriptions regarding the tibial neuromodulation terminal 3 may be found in the related descriptions below.

In some embodiments, by including the treatment parameter setting unit, the programming terminal may receive and display the scalp electrophysiological signal and the processing result of the scalp electrophysiological signal, allowing the doctor to input an individualized treatment parameter, and transmit the treatment parameter to the tibial neuromodulation terminal. The doctor can flexibly adjust the treatment parameter according to real-time data and feedback from the patient to ensure the best effect of the treatment plan.

In some embodiments, the tibial neuromodulation terminal 3 is further configured to, in response to determining that the abnormal signal reverts to a non-abnormal signal indicative of normal bladder activity, automatically terminate the tibial nerve electrical stimulation in response to determining that the non-abnormal signal and according to a preset delay time.

In some embodiments, the programming terminal 2 may determine the preset delay time based on a conversion confidence level by querying a fourth preset table. The fourth preset table may include a relationship between the conversion confidence level and the preset delay time. In some embodiments, the fourth preset table may be preset by the processor based on the default settings.

The conversion confidence level refers to the probability of the abnormal signal of the scalp electrophysiological signals appearing again within a preset time period. The preset time period refers to a period after a time point at which the scalp electrophysiological signal reverts to normal. In some embodiments, the preset time period may be preset by the programming terminal 2 based on the default settings.

In some embodiments, the scalp electrophysiological signal may revert to an abnormal signal within a preset time period after treatment, and the programming terminal 2 may screen a plurality of pieces of historical monitoring processing information with a plurality of preset time periods of the same duration, and determine the conversion confidence level based on a count of signals that are abnormal within the scalp electrophysiological signal and a total count of pieces of the historical monitoring processing information among the plurality of pieces of historical monitoring processing information. If the scalp electrophysiological signal appears abnormal during the preset time period, it is considered that the scalp electrophysiological signal reverts from the normal signal to the abnormal signal again during the preset time period after treatment.

The historical monitoring processing information refers to a scalp electrophysiological signal in a historical treatment. In some embodiments, the scalp electrophysiological signal in the historical monitoring processing information may contain the abnormal signal, or may not contain the abnormal signal.

For example, the programming terminal 2 may designate a ratio of a count of times the scalp electrophysiological signal appears as an abnormal signal again within the preset time period to the count of pieces of historical monitoring processing information within the preset time period as the conversion confidence level.

In some embodiments, the programming terminal 2 may also be configured to determine the conversion confidence level based on the monitoring processing information; determine the symptom severity based on the historical abnormal data and the pathological data; and determine the preset delay time based on the symptom severity and the conversion confidence level.

In some embodiments, the programming terminal 2 may also determine the symptom severity based on the abnormal data, the user data, and the pathological data.

For example, the programming terminal 2 may construct a feature vector based on the abnormal data, the user data, and the pathological data, and determine the symptom severity by matching with a vector database.

In some embodiments, the programming terminal 2 may also construct the vector database based on the historical abnormal data, the historical user data, the historical pathological data, and the historical symptom severity. The vector database may include a relationship between the historical abnormal data, the historical user data, the historical pathological data, and the historical symptom severity. In some embodiments, the vector database may be constructed by those skilled based on the experience.

The preset delay time refers to a time period to wait for automatic termination of the electrical stimulation after the abnormal signal reverts to the non-abnormal signal.

In some embodiments, the programming terminal 2 may determine, based on the conversion confidence level and the symptom severity, the preset delay time by querying a fifth preset table. The fifth preset table may include a relationship between the conversion confidence level, the symptom severity, and the preset delay time. In some embodiments, the fifth preset table may be preset by the programming terminal 2 based on the default settings.

For example, if the symptom severity is greater than a symptom degree threshold, the greater the symptom severity is, the larger the increased amplitude of the programming terminal 2 with respect to the preset delay time is; if the symptom severity is less than or equal to the symptom degree threshold, the smaller the symptom severity is, the larger the decreased amplitude of the programming terminal 2 with respect to the preset delay time is.

In some embodiments, by introducing the user data and the patient state information, the symptom severity of the user can be better determined, thereby realizing dynamic adjustment of parameters according to an individual difference and a real-time state of the patient, avoiding overstimulation and ineffective treatment, improving the comfort of the patient, and adapting to daily activities and physiological needs of the patient, and enhancing the treatment experience.

In some embodiments, according to the conversion confidence level, the symptom severity, and the preset delay time determined based on the monitoring processing information, the historical abnormal data, and the pathological data, the programming terminal can more accurately determine whether a current signal truly reflects a change in the physiological state of the patient. A conversion signal with a high confidence level can more reliably trigger a treatment neuromodulation, ensuring that the treatment is initiated when necessary, and is appropriately delayed and terminated after the abnormal signal disappears, thereby avoiding premature or late intervention, and can more comprehensively assess the symptom severity of the patient to more accurately adjust the treatment parameter.

In some embodiments, as shown in FIG. 1, the tibial neuromodulation terminal 3 further includes a neuromodulation unit 32 and a treatment parameter unit 33. The neuromodulation unit 32 receives a neuromodulation instruction from the scalp electrophysiological monitoring terminal 1 via a third wireless transmission unit 31, and at the same time, the treatment parameter unit 33 obtains the treatment parameter configured by the programming terminal 2, and initiates the tibial nerve electrical stimulation based on the neuromodulation instruction and the treatment parameter, and applies a corresponding electrical stimulation pulse to the patient.

From the above, it can be seen that the neuromodulation process of the closed-loop brain-computer interface tibial neuromodulation system of the embodiment of the present disclosure is as follows:

When the monitoring treatment is required, first the scalp electrophysiological monitoring terminal 1 is worn on the head of the patient, and the scalp electrophysiological monitoring terminal 1 acquires the scalp electrophysiological signal via the scalp-mounted electrode, processes the signal that is collected, and transmits a raw scalp electrophysiological signal and the processing result of the scalp electrophysiological signal to the programming terminal 2. The programming terminal 2 displays the scalp electrophysiological signal and the processing result of the scalp electrophysiological signal, and at the same time analyzes, processes, and displays the scalp electrophysiological signal that is received. The doctor configures, based on a display result of the raw data storage unit 22 and a display result of the signal analysis unit 23, suitable configuration information through the feature identification setting unit 24 and the feature value set setting unit 25. The programming terminal 2 transmits the suitable configuration information to the scalp electrophysiological monitoring terminal 1. The scalp electrophysiological monitoring terminal 1 determines whether the abnormal signal exists based on the processing result of the scalp electrophysiological signal and the configuration information. In response to determining that the abnormal signal exists, the scalp electrophysiological monitoring terminal 1 transmits the neuromodulation instruction to the tibial neuromodulation terminal 3. In addition, the scalp electrophysiological monitoring terminal 1 also transmits information such as the abnormal signal determination result to the programming terminal 2. Then the doctor makes a comprehensive judgment based on all the information displayed at the programming terminal 2, gives the corresponding treatment parameter, and inputs the treatment parameter through the treatment parameter setting unit 26 (or may obtain the treatment parameter automatically generated by the model or the treatment parameter that is pre-stored). The programming terminal 2 finally transmits the treatment parameter to the tibial neuromodulation terminal 3 to initiate the treatment.

For example, the programming terminal 2 receives the input information entered by the clinician, generates the configuration information based on the input information, and transmits the configuration information to the scalp electrophysiological monitoring terminal 1. The scalp electrophysiological monitoring terminal 1 screens the processing result of the scalp electrophysiological signal based on the feature set configuration information in the configuration information to obtain the feature value set. The feature identification model in the configuration information is configured to identify the feature value set and determine whether the abnormal signal exists. It is understood that, in response to determining that the abnormal signal is identified, the tibial nerve electrical stimulation is automatically initiated via the tibial neuromodulation terminal 3, and then, in response to determining that the abnormal signal reverts to the non-abnormal signal, the tibial nerve electrical stimulation is automatically terminated via the tibial neuromodulation terminal 3. Of course, after the abnormal signal disappears, a delay time may be set to realize the delayed termination function of the electrical stimulation, thereby further ensuring the effect of the electrical stimulation therapy.

In some embodiments, the scalp electrophysiological monitoring terminal 1 further includes a first wireless transmission unit 15, the programming terminal 2 further includes the second wireless transmission unit 21, and the tibial neuromodulation terminal 3 further includes the third wireless transmission unit 31. The scalp electrophysiological monitoring terminal 1, the programming terminal 2, and the tibial neuromodulation terminal 3 are wirelessly transmitted via the first wireless transmission unit 15, the second wireless transmission unit 21, and the third wireless transmission unit 31, which can minimize restrictions on the patient during activities.

In summary, the present disclosure achieves at least the following technical effects:

First, the closed-loop brain-computer interface tibial neuromodulation system of the embodiments of the present disclosure may monitor the scalp electrophysiological signal of the patient in real-time and automatically identify the signal. If a potential abnormal signal related to the bladder overactivity, such as the somatosensory evoked potential, the emotion evoked potential, and the action evoked potential that induces the urination response, is identified, the tibial nerve electrical stimulation is initiated, and achieve an on-demand stimulation and the on-demand neuromodulation during the persistence period of the overactive bladder, which is smarter and more accurate than a traditional open-loop stimulation.

Second, the closed-loop brain-computer interface tibial neuromodulation system of the embodiments of the present disclosure displays the information such as the scalp electrophysiological signal, the signal processing result, and the abnormal signal determination result via the programming terminal, so that the clinician can configure the individualized treatment parameter in the targeted manner based on the above displayed information. The programming terminal transmits the individualized treatment parameter to the tibial neuromodulation terminal, thereby improving the treatment accuracy and effectively alleviating patient symptoms, which is an embodiment of precision medicine.

Third, differing from conventional approaches with imprecise universal neuromodulation and continuous electrical stimulation causing patient discomfort, while considering that the tibial nerve may entry into a refractory period during sustained stimulation, the closed-loop brain-computer interface tibial neuromodulation system of the present disclosure can automatically initiate and terminate the electrical stimulation according to a monitoring result of an individual scalp electrophysiological signal, realize the on-demand stimulation, an intermittent stimulation, and a quantitative precise stimulation, effectively avoid the ineffective stimulation during the refractory period, and significantly reduce the discomfort caused by stimulation to the patient.

It should be noted that, in the present disclosure, unless otherwise expressly specified and limited, the terms “connection”, “fixed” and other terms should be understood in a broad sense, for example, it may be a fixed connection, a removable connection, or one integrated unit; it may be a mechanical connection, an electrical connection, or one that is communicable with each other; it may be a direct connection or an indirect connection through an intermediate medium, and it may be a connectivity within the two elements or an interactive relationship of the two elements, unless otherwise expressly limited. For those skilled in the art, the specific meanings of the above terms in the present disclosure may be understood on a case-by-case basis.

It should also be noted that the terms “include”, “comprises”, or any other variant thereof, are intended to cover the following non-exclusive inclusion, such that a process, method, commodity, or apparatus comprising a set of elements includes not only those elements, but also other elements that are not explicitly listed, or that are inherent to such process, method, commodity, or apparatus. In the absence of further limitation, the elements qualified by the statement “including a . . . ” do not preclude the existence of another identical element in the process, method, commodity, or apparatus that includes said element.

The foregoing is merely an example of an embodiment of the present disclosure and is not intended to limit the present disclosure. The present disclosure is subject to various changes and variations for those skilled in the art. Any modifications, equivalent substitutions, improvements, etc., which are made within the spirit and principles of the present disclosure should be included in the scope of the claims of the present disclosure.

What is claimed is:

1. A closed-loop brain-computer interface tibial neuromodulation system, wherein the closed-loop brain-computer interface tibial neuromodulation system comprises: a scalp electrophysiological monitoring terminal, a tibial neuromodulation terminal, and a programming terminal, the scalp electrophysiological monitoring terminal is connected to the tibial neuromodulation terminal, and the programming terminal is connected to the tibial neuromodulation terminal;
the scalp electrophysiological monitoring terminal is configured to monitor a scalp electrophysiological signal in real-time via a non-invasive manner, and to identify an abnormal signal indicative of bladder overactivity upon an occurrence of a urinary urgency symptom of a patient, wherein the scalp electrophysiological signal indirectly characterizes frequency bands related to the bladder overactivity upon the occurrence of the urinary urgency symptom of the patient;
the tibial neuromodulation terminal is configured to, in response to detecting the abnormal signal, initiate a tibial nerve electrical stimulation to achieve an on-demand neuromodulation for the patient during a symptom onset and persistence period of the bladder overactivity;
the programming terminal is configured to obtain a treatment parameter related to the on-demand neuromodulation;
the closed-loop brain-computer interface tibial neuromodulation system further comprises: configuration information, wherein the configuration information is configured to be generated by the programming terminal and transmitted to the scalp electrophysiological monitoring terminal;
the scalp electrophysiological monitoring terminal is further configured to identify whether the abnormal signal exists via the scalp electrophysiological signal monitored in real-time and the configuration information; the configuration information at least includes feature set configuration information and a feature identification model, the feature set configuration information being configured to screen a processing result of the scalp electrophysiological signal, the feature identification model being configured to identify whether the abnormal signal exists based on the processing result of the scalp electrophysiological signal;
the tibial neuromodulation terminal is further configured to, in response to determining that the abnormal signal reverts to a non-abnormal signal indicative of normal bladder activity, automatically terminate the tibial nerve electrical stimulation according to a preset delay time;
the programming terminal is further configured to obtain input information entered by a clinician and generate the configuration information based on the input information;
the programming terminal includes a feature identification setting unit, the scalp electrophysiological monitoring terminal includes a feature identification unit;
the feature identification setting unit is configured to receive model input information within the input information, generate the feature identification model based on the model input information, and transmit the feature identification model to the feature identification unit;
the programming terminal includes a feature value set setting unit, the scalp electrophysiological monitoring terminal includes a feature value set configuration unit;
the feature value set setting unit is configured to receive set input information within the input information, generate the feature set configuration information based on the set input information, and transmit the feature set configuration information to the feature value set configuration unit;
the feature value set configuration unit is configured to screen the processing result of the scalp electrophysiological signal according to the feature set configuration information and determine a feature value set, the feature value set at least including feature values under an overactive bladder during an awake activity state of the patient, and feature values under the overactive bladder during a transitional sleep state of the patient; and
the feature identification unit is configured to identify the feature value set based on the feature identification model and determine whether the abnormal signal exists.

2. The closed-loop brain-computer interface tibial neuromodulation system according to claim 1, wherein the programming terminal further includes a raw data storage unit and a signal analysis unit;
the raw data storage unit is configured to receive and display the scalp electrophysiological signal, the processing result of the scalp electrophysiological signal, and an abnormal signal determination result; and
the signal analysis unit is configured to process the scalp electrophysiological signal and output and display a programming-processed signal.

3. The closed-loop brain-computer interface tibial neuromodulation system according to claim 2, wherein the programming terminal further includes a treatment parameter setting unit;
the treatment parameter setting unit is configured to, in response to determining that the raw data storage unit receives the scalp electrophysiological signal, the processing result of the scalp electrophysiological signal, and the abnormal signal determination result, receive the treatment parameter that is individualized input by the clinician; and
transmit the treatment parameter to the tibial neuromodulation terminal, to achieve the on-demand neuromodulation for the patient during the symptom onset and persistence period of the bladder overactivity according to the treatment parameter after automatic initiation of the tibial nerve electrical stimulation.

4. The closed-loop brain-computer interface tibial neuromodulation system according to claim 3, wherein the treatment parameter includes at least one of: stimulation waveform, amplitude, pulse width, frequency, rise cycle, fall cycle, duration cycle, treatment duration, frequency modulation or constant frequency, voltage or current source.

5. The closed-loop brain-computer interface tibial neuromodulation system according to claim 1, wherein the scalp electrophysiological monitoring terminal further includes an acquisition unit, a filtering unit, and a feature extraction unit,
the acquisition unit is configured to acquire the scalp electrophysiological signal on a head via a scalp-mounted electrode; and
the feature extraction unit is configured to perform cluster analysis on the scalp electrophysiological signal processed by the filtering unit according to specific frequency bands, and calculate feature values within the specific frequency bands as the processing result of the scalp electrophysiological signal; the specific frequency bands include a short-time spectral energy and a change rate of θ waves within a 3.5-8 Hz bandpass, a short-time spectral energy and a change rate of α waves within an 8-12 Hz bandpass, and a short-time spectral energy and a change rate of β waves within a 12-33 Hz bandpass.

6. The closed-loop brain-computer interface tibial neuromodulation system according to claim 1, wherein the scalp electrophysiological monitoring terminal is further configured to:
extract first scalp electrophysiological feature values under a non-overactive bladder during the awake activity state and the transitional sleep state of the patient through a pre-wearing usage during a predefined period;
extract second scalp electrophysiological feature values under the overactive bladder during the awake activity state and the transitional sleep state of the patient;
determine a scalp electrophysiological feature set for the pre-wearing usage based on the first scalp electrophysiological feature values and the second scalp electrophysiological feature values; and
perform a comparative analysis between the scalp electrophysiological feature set during the pre-wearing usage and a feature value set of an electrophysiological signal monitored during a formal wearing usage, to determine whether a requirement for initiating an electrical stimulation therapy is met to perform an individualized pre-configuration for the patient.
